(19) United States
(12) Patent Application Publication
(10) Pub. No.: US 2010/0036329 A1
(43) Pub. Date: Feb. 11, 2010

(54) HEMOSTASIS VALVE

(76) Inventor: Nasser Razack, Bradenton, FL (US)

Correspondence Address: Maxey Law Offices, PLLC, 13630 58TH ST. NORTH, SUITE 101, CLEARWATER, FL, 33760, US

(21) Appl. No.: 12/187,547

(22) Filed: Aug. 7, 2008

(51) Int. Cl. A61M 39/22

(52) U.S. Cl. 604/256

(57) ABSTRACT

A hemostasis valve to maintain hemostasis during the insertion, removal or reposition of a medical instrument in the vascular system of a patient. The hemostasis valve includes a valve body having lumen extending therethrough, a first valve positioned across the lumen, and having a passage with a diameter that is continuously variable, a second valve positioned across the lumen, and having a passage with a diameter that is continuously variable. The first and second valves being spaced apart longitudinally along the lumen. A connector rotatably attached to the valve body for connecting the hemostasis valve to another instrument. The first and second valves are selectively operable to create an opening through the passage respectively of a diameter to permit admittance of at least one intravascular instrument through at least one of the first and second valves while maintaining hemostasis. The second valve is rotatably attached to the valve body without effecting the diameter of the passage therethrough.

### FILED OF THE INVENTION

The present invention relates generally to an apparatus that can be used to limit or prevent the loss of bodily fluids from a patient when an access device is introduced into the body of patient, and more particularly, relating to a hemostasis valve for use in medical procedures.

### BACKGROUND OF THE INVENTION

Hemostasis valves are well known and used in medical procedures requiring the insertion of a catheter or other device into the vascular system of a patient.

Hemostasis valves are used for leak-proof introduction of catheters or other devices into the vascular system or elsewhere in the body of a patient. Typically, a guide catheter is connected to the distal end of the hemostasis valve, and an operating instrument is inserted into the proximal end and through the guide catheter to the desired location in the patient. Once the operating instrument is in place, the valve is closed around the instrument to prevent blood from escaping from the body of the patient.

One of the problems found in conventional hemostasis valves is the reposition, the removal and insertion of another medical instrument, or the insertion of multiple medical instruments through a single guide catheter at different times during a surgical procedure. Each time an instrument is inserted or removed, and in some prior hemostasis valves, repositioned, the valve must be opened to allow for the passage of the instrument through the valve. During the time the valve is open, hemostasis is lost and vast amounts of blood and other fluids are capable of being lost from the patient.

Accordingly, there is a need for a new hemostasis valve that permits the reposition, removal and insertion of medical instruments into the vascular system of a patient through a catheter or other medical device, while maintaining hemostasis and preventing blood loss from the patient.

### SUMMARY OF THE INVENTION

The embodiments of the present invention addresses this need by providing a hemostasis valve that overcomes the disadvantages of the prior art hemostasis valves. The hemostasis valve in accordance with the present invention can be used in a variety of diagnostic, therapeutic, and interventional procedures.

To achieve these and other advantages, in general, in one aspect, a hemostasis valve for preventing blood loss while permitting the introduction of intravascular instruments is provided. The hemostasis valve includes a valve body having a lumen extending therethrough, a first valve positioned across the lumen that is operable to selectively permit fluid flow through the lumen in an open position or preclude fluid flow through the lumen in a closed position, a second valve positioned across the lumen that is operable to selectively permit fluid flow through the lumen in an open position or preclude fluid flow through said lumen in a closed position, a connector rotatably connected to the valve body for connecting the hemostasis valve to another instrument. The first and said second valves are longitudinally spaced along the lumen. The first and said second valves are selectively operable to create an opening of a diameter to permit admittance of intravascular instruments through at least one of the first and second valves while maintaining hemostasis by a sliding seal.

In general, in another aspect, the second valve is rotatably connected at one end thereof to the valve body for rotation about the valve body without effecting the opening or closing of the second valve.

In general, in another aspect, a hemostasis valve for preventing blood loss while permitting the introduction of intravascular instruments is provided. The hemostasis valve includes a valve body having a proximal end, a distal end, and lumen extending through the valve body, the lumen capable of receiving for passage therethrough at least one intravascular instrument. A first valve positioned across the lumen, and having a passage with a diameter that is continuously variable, the first valve is operable for the controllable opening and closing of the passage thereof to selectively permit fluid flow through the lumen in an open position or preclude fluid flow through the lumen in a closed position. A second valve positioned across the lumen, and having a passage with a diameter that is continuously variable, the second valve is operable for the controllable opening and closing of the passage thereof to selectively permit fluid flow through the lumen in an open position or preclude fluid flow through the lumen in a closed position. The first and the second valves being spaced apart longitudinally along the lumen. A connector rotatably attached to the distal end of the valve body for connecting the hemostasis valve to another instrument. The first and the second valves are selectively operable to create an opening through the passage respectively of a diameter to permit admittance of at least one intravascular instrument through at least one of the first and second valves while maintaining hemostasis.

In general, in another aspect, a hemostasis valve for preventing blood loss while permitting the introduction of intravascular instruments is provided. The hemostasis valve includes a valve body having a proximal end, a distal end, and lumen extending through the valve body, the lumen capable of receiving for passage therethrough at least one intravascular instrument, a valve positioned across the lumen intermediate of the proximal and distal ends, and having a passage with a diameter that is continuously variable, the first valve is operable for the controllable opening and closing of the passage thereof to selectively permit fluid flow through the lumen in an open position or preclude fluid flow through the lumen in a closed position, a first connector at the proximal end for connecting another instrument to the proximal end of the valve body, and a second connector at the distal end rotatably connected to the valve body, and for connecting another instrument to the distal end of the valve body.

In general, in another aspect, a hemostasis valve for preventing blood loss while permitting the introduction of intravascular instruments is provided. The hemostasis valve includes a valve body having a proximal end, a distal end, an intermediate barrel portion, and lumen extending through the valve body, the lumen capable of receiving for passage therethrough at least one intravascular instrument; a first compressible seal having a normally open passage therethrough positioned within the valve body across the lumen, and which is selectively closeable when deformed by exerting a compressive force on the compressible seal; a first rotatable locking nut joined to the valve body and forming a first valve assembly therewith which together encloses the first compressible seal within the first valve assembly, the first rotatable locking nut being rotatable about the valve body to selectively exert and remove compressive force applied to the compressible seal so as to selectively close and open the passage thereof about the circumference of an intravascular instrument in response to the exertion and removal of the force respectively; a second compressible seal having a normally open passage therethrough positioned within the valve body across the lumen at a longitudinally spaced distance from the first compressible seal, and which is selectively closeable when deformed by exerting a compressive force on the compressible seal; a second rotatable locking nut having a proximal end connected to the valve body, and a distal end connected to the intermediate barrel portion and forming a second valve assembly therewith which together encloses the second compressible seal within the second valve assembly; the second rotatable locking nut being rotatable about the barrel portion to selectively exert and remove compressive force applied to the second compressible seal so as to selectively close and open the passage thereof about the circumference of an intravascular instrument in response to the exertion and removal of the force respectively; the proximal end of the second rotatable locking nut being rotatably connected to the valve body for rotation about the valve body without effecting rotation of the second rotatable locking nut about the barrel portion; a connector rotatably attached to the distal end of the valve body for connecting the hemostasis valve to another instrument; and wherein each passage of the first and the second compressible seals are selectively and opened and closed to permit admittance of at least one intravascular instrument through at least one of the passage of the first and second compressible seals while maintaining hemostasis.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The accompanying drawings, which are included to provide further understanding of the invention and are incorporated in and constitute a part of this specification, illustrate preferred embodiments of the invention and together with the description serve to explain the principles of the invention, in which:

FIG. 1 is an elevation view of an embodiment of a hemostasis valve in accordance with the principles of the present invention;

FIG. 2 is an enlarged cross section of the hemostasis valve of FIG. 1 through a first valve;

FIG. 3 is an enlarged cross section of the hemostasis valve of FIG. 1 through a second valve;

FIG. 4 is an elevation view of a second embodiment of a hemostasis valve in accordance with the principles of the present invention;

FIG. 5 is an elevation view of a third embodiment of a hemostasis valve in accordance with the principles of the present invention; and

FIG. 6 is an enlarged cross section of the hemostasis valve of FIG. 5.

### DETAILED DESCRIPTION OF THE INVENTION

Referring now to the drawings, the same reference numerals are used to identify like parts of the illustrated embodiments. The present invention relates to an improved hemostasis valve apparatus that minimizes the loss of body fluids during insertion, repositioning or removal of intravascular medical instruments.

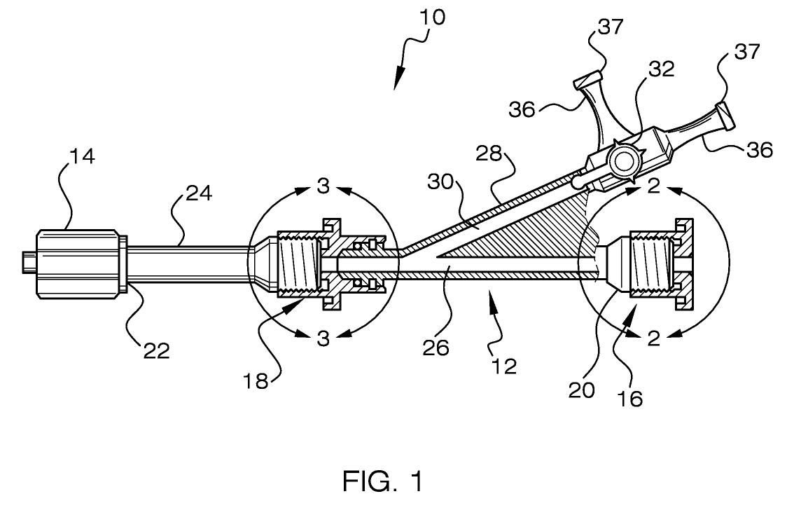

Referring now to FIG. 1, which illustrates a hemostasis valve 10 according to the present invention. The hemostasis valve 10 includes a valve body 12, a rotatable connector 14, a first valve 16, and a second valve 18. The first and second valves 16 and 18 are longitudinally spaced along the valve body 12. The valve body 12 has a proximal end 20, an opposing distal end 22, an intermediate barrel portion 24, and a through-lumen 26. Rotatable connector 14 is positioned at the distal end 22 of the valve body 12. Rotatable connector 14 is an example of a means for rotatably coupling said valve body 12 in fluid communication with a catheter or other medical device. The rotatable connector 14 may be a rotatable luer lock male connector, which such connector is well known in the field of the invention. The through-lumen 26 is capable of receiving, for passage therethrough, an intravascular instrument (not shown) for insertion into the vascular system of a patient through the introducer that is attachable to rotatable connector 14.

A supplemental access or injection tube 28 may be attached to the valve body 12. The injection tube 28 includes a branch lumen 30 formed therethrough so as to be in fluidic communication with said through lumen 26 at a position intermediate the first and second valves 16 and 18. The injection tube can be used to introduce fluids into the body of the patient. A stopcock, such as a two-way stopcock 32 may be attachable or integrated with the proximal end 34 of the injection tube 28 to selectively control fluid flow into the branch lumen 30 from one or more connection ports 36. The connection ports 36 may be fitted with connectors 37 permitting the connection of other devices to the connection ports.

The first and second valves 16 and 18 are positioned across the lumen 26, and each are independently operable to selectively permit fluid flow through the lumen in an open position or preclude fluid flow through the lumen in a closed position. The first and second valves 16 and 18 are selectively operable to create an opening of a diameter to permit admittance of intravascular instruments through at least one of the first and second valves while maintaining hemostasis by a sliding seal with an intravascular instrument.

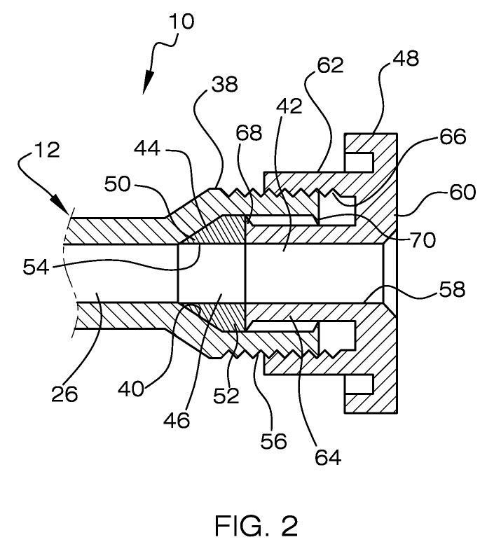

In FIG. 2, there is shown an enlarged section of the valve body 12 through the first valve 16. The first valve 16 includes a valve assembly 38 having an interior surface 40 defining a lumen 42 therethrough, a compressible seal 44 defining a passage 46 therethrough having a diameter that is continuously variable, and a hollow, rotating locking nut 48 for selectively compressing the seal 44 to controllably open and close the passage 46. The compressible seal 44 has a hollow conical distal end 50 extending from a hollow, cylindrical proximal end 52. Compressible seal 44 has an interior surface 54 defining the passage 46. Distal end 50 is configured to mate within a corresponding distal portion of interior surface 40. It can be appreciated that the exterior surface of hollow, compressible seal 44 may have other configurations as long as both compressible seal 44 and interior surface 40 of configured to cooperate.

Compressible seal 44 responds to compressive forces exerted thereon to seal valve 16. When compressive force is exerted on compressible seal 44, a portion of compressible seal 44 moves radially inward to form a progressively tighter seal around an elongate instrument such as a catheter or guide wire that is disposed in compressive seal 44. The amount of compressive force that is being exerted on compressible seal 44 can be incrementally adjusted so that a seal is formed or maintained around the catheter or guide wire while still allowing the catheter or guide wire to be repositioned or even removed without having to remove all of the compressive force acting on the compressible seal. Also, because of the adjustability of seal 44 it is possible to dispose elongate medical instruments having a variety of different diameters therethrough.

Hollow, rotatable locking nut 48 is configured to selectively thread onto engagement threads 56 of valve body 12 and to selectively compress compressible seal 44. Rotatable locking nut 48 has an interior surface 58 defining an opening therethrough. Locking nut 48 includes an end wall 60, an outer, annular side wall 62 extending integrally and distally from end wall 60, and an inner, hollow, annular neck 64 extending integrally and distally from end wall 60. Extending from annular side wall 62 is a set of engagement threads 66 configured for rotational, threaded engagement with engagement threads 56 on the proximal end of valve body 12.

Neck 64 has an annular ridge 68 extending from a distal end thereof which, during assembly, slips over an annular ridge 70 on the proximal end of the valve body 12, and prevents the rotatable locking nut 48 from being inadvertently threaded off valve body 12. Thus, in order to remove the rotatable locking nut 48 from the valve body 12, the nut must be pulled from the body such that ridge 66 is pulled over ridge 70.

In use, rotatable locking nut 48 may be selectively rotated relative to valve body 12 such that neck 64 proceed distally to compress against compressible seal 44, causing compressible seal 44 to close. Upon desiring to open compressible seal 44, rotatable locking nut 48 is rotated proximally, thereby opening seal 44.

Valve 16 is one example of a structure capable of performing the function of sealing lumen 26. It is appreciated that other structures could be substituted for the structure of valve 16 to facilitate the selective opening and closing of the lumen 26 permitting the insertion of an intravascular instrument through the lumen to maintaining hemostasis.

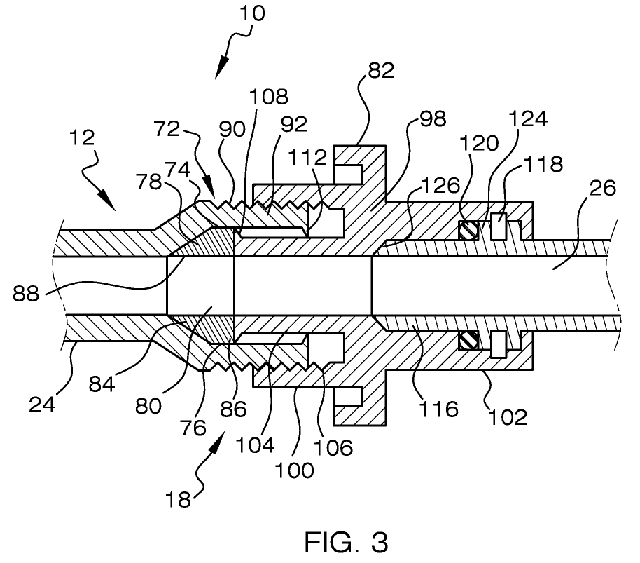

In FIG. 3, there is shown an enlarged section of the valve body 12 through the second valve 18. Similarly to the first valve 16, the second valve 18 includes a valve assembly 72 having an interior surface 74 defining a lumen 76 therethrough, a compressible seal 78 defining a passage 80 therethrough having a diameter that is continuously variable, and a hollow, rotating locking nut 82 for selectively compressing the seal 78 to controllably open and close the passage 80. The compressible seal 78 has a hollow conical distal end 84 extending from a hollow, cylindrical proximal end 86. Compressible seal 78 has an interior surface 88 defining the passage 80. Distal end 84 is configured to mate within a corresponding distal portion of interior surface 74. It can be appreciated that the exterior surface of hollow, compressible seal 78 may have other configurations as long as both compressible seal 78 and interior surface 74 of configured to cooperate.

Compressible seal 78 responds to compressive forces exerted thereon to seal valve 18. When compressive force is exerted on compressible seal 78, a portion of the compressible seal moves radially inward to form a progressively tighter seal around an elongate instrument such as a catheter or guide wire that is disposed in the compressive seal. The amount of compressive force that is being exerted on the compressible seal 78 can be incrementally adjusted so that a seal is formed or maintained around the catheter or guide wire while still allowing the catheter or guide wire to be repositioned or even removed without having to remove all of the compressive force acting on the compressible seal. Also, because of the adjustability of compressible seal 78 it is possible to dispose elongate medical instruments having a variety of different diameters therethrough.

Hollow, rotatable locking nut 82 is configured to selectively thread onto engagement threads 90 extending from proximal end 92 of the barrel portion 24 and to selectively compress compressible seal 78. Locking nut 82 includes an end wall 98, an first outer, annular side wall 100 extending integrally and distally from end wall 98, an second outer annular side wall 102 extending integrally and proximally from end wall 98, and an inner, hollow, annular neck 104 extending integrally and distally from end wall 98. Extending from annular side wall 100 is a set of engagement threads 106 configured for rotational, threaded engagement with engagement threads 90 on the proximal end 92 of the barrel portion 24.

Neck 104 has an annular ridge 108 extending from a distal end thereof which, during assembly, slips over an annular ridge 112 on the proximal end 92 of the barrel portion 24, and prevents the rotatable locking nut 82 from being inadvertently threaded off the barrel portion. Thus, in order to remove the rotatable locking nut 82 from the barrel portion 24, the nut must be pulled from the body such that ridge 108 is pulled over ridge 112.

The proximal end 114 of the rotatable locking nut 82 is rotatably connected to an intermediate stub portion 116 of the valve body 12 for rotation about the valve body without causing the nut 82 to threadably rotate about the barrel portion 24 and effecting the seal of valve 18. Further, it is important to note, the proximal end 114 of the rotatable locking nut 82 is rotatable about the valve body 12 separate from the valve body being rotated. In other words, nut 82 can be rotated while holding the valve body 12 against rotation. The second outer annular side wall 102 of the rotatable lucking nut 82 is capable of rotatably receiving the stub portion 116 therein. The stub portion 116 can be rotatably retained within the annual side wall 102 either by over molding techniques, by a snap ring 118 or by another suitable means. A seal 120 is received in an annular space between the end wall 82 and a annular ridge 124 on the stub portion 116. The stub portion 116 has a conical distal end 126 that is configured to mate with the proximal side of the end wall 98.

In use, rotatable locking nut 82 may be selectively rotated relative to valve body 12, including the barrel portion 24, such that neck 104 proceed distally to compress against compressible seal 78, causing compressible seal 78 to close. Upon desiring to open compressible seal 78, rotatable locking nut 82 is rotated proximally, thereby opening seal 78.

As in the first valve 16, the second valve 18 is one example of a structure capable of performing the function of sealing lumen 26. It is appreciated that other structures could be substituted for the structure of valve 18 to facilitate the selective opening and closing of the lumen 26 permitting the insertion of an intravascular instrument through the lumen to maintaining hemostasis.

It can now be understood, in one advantage of the hemostasis valve 10, an intravascular instrument can be introduced, repositioned, or removed from the hemostasis valve while maintaining hemostasis and minimizing blood loss from the patient. In this regard, the first and the second valves 16 and 18 are selectively operable to create an opening of a diameter to permit admittance of intravascular instruments through at least one of the valves while maintaining hemostasis by a sliding seal.

In one particular example, after a catheter or medical device (not shown) attached to the rotatable coupling 14 has been placed in a patient's vascular system, the second valve 18, which is closest to the point of entry into the patient's vascular system, is closed to preclude fluid flow through lumen 26. This allows the opening of the first valve 16, which is furthest from the entry point, without blood loss since the second valve 18 is closed. An intravascular instrument can then be inserted through the passage 46 of the first valve 16 and into the lumen 26 of the valve body 12 in the space between first and second valves 16 and 18. The first valve 16 can then be closed to provide sufficient friction against the intravascular instrument to providing a sliding seal between the instrument and the valve to maintain hemostasis. Now with the intravascular instrument in the lumen 26 between the valves, and with the first valve 16 closed, the second valve 18 (closest to the patient) can be opened to permit fluid flow through the lumen 26 distally. The intravascular instrument can now be advanced further through the lumen 26, and through passage 80 of the second valve and into the introducer to be positioned in the patient while hemostasis is maintained with the first valve 16 closed. If desired, the second valve 18 may also be closed to provide sufficient friction against the intravascular instrument to provide a sliding seal between the instrument and the valve to maintain hemostasis.

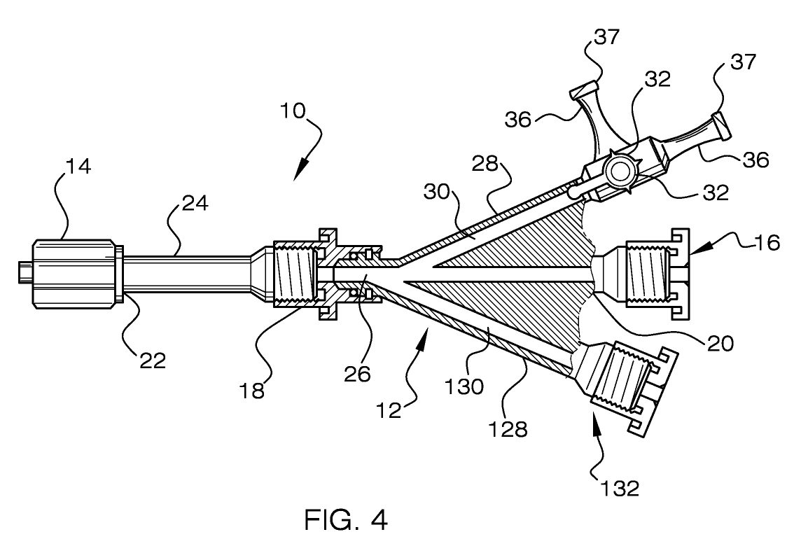

While one configuration of the hemostasis valve 10 is described above, it can be appreciated that the hemostasis valve in accordance with the principals of the present invention may have various other configurations. An example of another possible configuration is shown in FIG. 4.

In FIG. 4, the above discussed embodiments of the hemostasis valve 10 in accordance to the present invention can have more than one port for the insertion of an intravascular instrument into the vascular system of a patient. In this embodiment, the valve 10 is exactly the same as discussed above, but further includes a secondary or supplemental insertion tube 128 attached to the valve body 12. The supplemental insertion tube 128 includes a branch lumen 130 in fluidic communication with the through lumen 26 at a position intermediate valves 16 and 18. A third valve 132 is positioned across the branch lumen 130.

Like valves 16 and 18, valve 132 is operable to selectively permit fluid flow through lumen 130 in an open position or preclude fluid flow through lumen 130 in a closed position. Valve 132 is selectively operable to create an opening of a diameter to permit admittance of intravascular instruments therethrough while maintaining hemostasis by a sliding seal with an intravascular instrument. Valve 132 can be identical in structure and operation as valve 16 as described above, and as such a detailed discussion of valve 132 is not need.

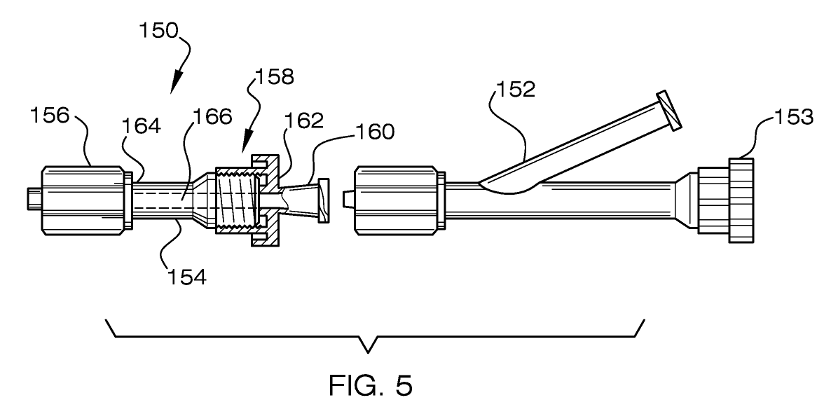

In FIG. 5, there is shown another embodiment of a hemostasis valve 150 in accordance with the principals of the present invention. In this embodiment, the hemostasis valve 150 is to be connected between another medical device, such as Y-adaptor 152 having a hemostasis valve 153 and an introducer (not shown) to prevent blood loss from a patient that otherwise would not be possible with just the Y-adaptor alone. The hemostasis valve 150 includes a valve body 154, a rotatable connector 156, a valve 158, and connector 160. The valve body 154 has a proximal end 162, an opposing distal end 164 and a through-lumen 166. Rotatable connector 156 is positioned at the distal end 164. Rotatable connector 156 is an example of a means for rotatably coupling the valve body 154 in fluid communication with an introducer (not shown). The rotatable connector 156 may be a rotatable luer lock male connector, which such connector is well known in the field of the invention. The through-lumen 166 is capable of receiving, for passage therethrough, an intravascular instrument (not shown) for insertion into the vascular system of a patient through the introducer that is attachable to rotatable connector 156.

Valve 158 is positioned across lumen 166, and is operable to selectively permit fluid flow through the lumen in an open position or preclude fluid flow through the lumen in a closed position. Valve 158 is selectively operable to create an opening of a diameter to permit admittance of intravascular instruments through at least one of the first and second valves while maintaining hemostasis by a sliding seal with an intravascular instrument.

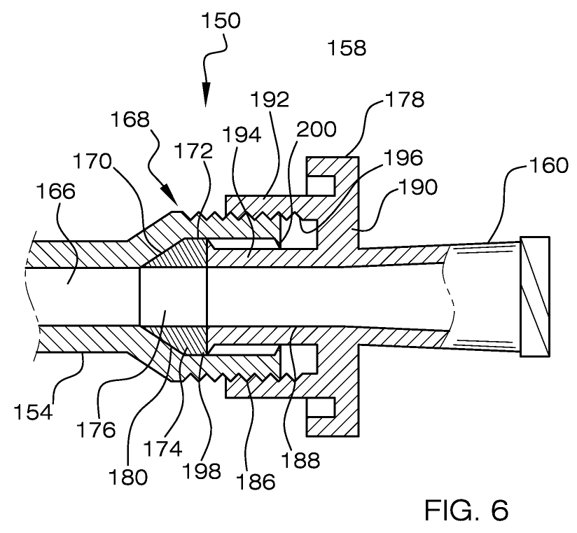

In FIG. 6, there is shown an enlarged section of the valve body 154 through valve 158. Valve 158 includes a valve assembly 168 having an interior surface 170 defining a lumen 172 therethrough, a compressible seal 174 defining a passage 176 therethrough having a diameter that is continuously variable, and a hollow, rotating locking nut 178 for selectively compressing the seal 174 to controllably open and close passage 176. Compressible seal 174 has a hollow conical distal end 180 extending from a hollow, cylindrical proximal end 182. Compressible seal 174 has an interior surface 184 defining the passage 176. Distal end 180 is configured to mate within a corresponding distal portion of interior surface 170.

It can be appreciated that the exterior surface of hollow, compressible seal 174 may have other configurations as long as both compressible seal 174 and interior surface 170 of configured to cooperate.

Compressible seal 174 responds to compressive forces exerted thereon to seal valve 158. When compressive force is exerted on compressible seal 174, a portion of compressible seal 174 moves radially inward to form a progressively tighter seal around an elongate instrument such as a catheter or guide wire that is disposed in compressive seal 174. The amount of compressive force that is being exerted on compressible seal 174 can be incrementally adjusted so that a seal is formed or maintained around the catheter or guide wire while still allowing the catheter or guide wire to be repositioned or even removed without having to remove all of the compressive force acting on the compressible seal. Also, because of the adjustability of seal 174 it is possible to dispose elongate medical instruments having a variety of different diameters therethrough.

Hollow, rotatable locking nut 178 is configured to selectively thread onto engagement threads 186 of valve body 154 and to selectively compress compressible seal 174. Rotatable locking nut 178 has an interior surface 188 defining an opening therethrough. Locking nut 178 includes an end wall 190, an outer, annular side wall 192 extending integrally and distally from end wall 190, and an inner, hollow, annular neck 194 extending integrally and distally from end wall 190. Extending from annular side wall 190 is a set of engagement threads 196 configured for rotational, threaded engagement with engagement threads 186 on valve body 154.

Neck 194 has an annular ridge 198 extending from a distal end thereof which, during assembly, slips over an annular ridge 202 on the proximal end of the valve body 154, and prevents the rotatable locking nut 178 from being inadvertently threaded off valve body 154. Thus, in order to remove the rotatable locking nut 178 from the valve body 154, the nut must be pulled from the body such that ridge 198 is pulled over ridge 200.

Extending distally from end wall 190 is connector 160 for attaching valve body 154 to another medical instrument, such as the Y-adaptor 152. Connector 160 can be any standard connector used to interconnect medical devices together, such as a luer type connector.

In use, rotatable locking nut 178 may be selectively rotated relative to valve body 154 such that neck 194 proceed distally to compress against compressible seal 174, causing compressible seal 174 to close. Upon desiring to open compressible seal 174, rotatable locking nut 178 is rotated proximally, thereby opening seal 174.

Valve 158 is one example of a structure capable of performing the function of sealing lumen 26. It is appreciated that other structures could be substituted for the structure of valve 16 to facilitate the selective opening and closing of the lumen 26 permitting the insertion of an intravascular instrument through the lumen to maintaining hemostasis.

A number of embodiments of the present invention have been described. Nevertheless, it will be understood that various modifications may be made without departing from the spirit and scope of the invention. Accordingly, other embodiments are within the scope of the following claims.

1. A hemostasis valve for preventing blood loss while permitting the introduction of intravascular instruments, the hemostasis valve comprising:
a valve body having a lumen extending therethrough;
a first valve positioned across said lumen that is operable to selectively permit fluid flow through said lumen in an open position or preclude fluid flow through said lumen in a closed position;
a second valve positioned across said lumen that is operable to selectively permit fluid flow through said lumen in an open position or preclude fluid flow through said lumen in a closed position;
a connector rotatably connected to said valve body for connecting the hemostasis valve to another instrument;
wherein said first and said second valves are longitudinally spaced along said lumen; and
wherein said first and said second valves are selectively operable to create an opening of a diameter to permit admittance of intravascular instruments through at least one of said first and second valves while maintaining hemostasis by a sliding seal.

2. The hemostasis valve of claim 1, wherein said second valve is rotatably connected at one end thereof to said valve body for rotation about said valve body without effecting the opening or closing of said second valve.

3. A hemostasis valve for preventing blood loss while permitting the introduction of intravascular instruments, the hemostasis valve comprising:
a valve body having a proximal end, a distal end, and lumen extending through said valve body, said lumen capable of receiving for passage therethrough at least one intravascular instrument;
a first valve positioned across said lumen, and having a passage with a diameter that is continuously variable, said first valve is operable for the controllable opening and closing of said passage thereof to selectively permit fluid flow through said lumen in an open position or preclude fluid flow through said lumen in a closed position;
a second valve positioned across said lumen, and having a passage with a diameter that is continuously variable, said second valve is operable for the controllable opening and closing of said passage thereof to selectively permit fluid flow through said lumen in an open position or preclude fluid flow through said lumen in a closed position;
said first and said second valves being spaced apart longitudinally along said lumen;
a connector rotatably attached to said distal end of said valve body for connecting the hemostasis valve to another instrument;
wherein said first and said second valves are selectively operable to create an opening through said passage respectively of a diameter to permit admittance of at least one intravascular instrument through at least one of said first and second valves while maintaining hemostasis.

4. The hemostasis valve of claim 3, wherein said valve body further comprises an intermediate barrel portion between said second valve and said distal end.

5. The hemostasis valve of claim 3, wherein said second valve is rotatably connected at one end thereof to said valve body for rotation about said valve body without effecting the diameter of said passage of said second valve.

6. The hemostasis valve of claim 3, wherein each of said valves are of the Touhy-Borst type.

7. A hemostasis valve comprising:
a valve body having a proximal end, a distal end, and lumen extending through said valve body, said lumen capable of receiving for passage therethrough at least one intravascular instrument;
a valve positioned across said lumen intermediate of said proximal and distal ends, and having a passage with a diameter that is continuously variable, said first valve is operable for the controllable opening and closing of said passage thereof to selectively permit fluid flow through said lumen in an open position or preclude fluid flow through said lumen in a closed position;
a first connector at said proximal end for connecting another instrument to said proximal end of said valve body; and
a second connector at said distal end rotatably connected to said valve body, and for connecting another instrument to said distal end of said valve body.

8. The hemostasis valve of claim 7, wherein said first connector is a luer lock female connector; and wherein said second connector is a rotatable luer lock male connector.

9. The hemostasis valve of claim 7, wherein said valve body farther includes an intermediate barrel portion between said valve and said second connector.

10. The hemostasis valve of claim 7, wherein said valve comprises:
a compressible seal that defines said passage, said passage being normally open; and
a rotatable locking nut joined to said valve body and forming a valve assembly therewith which together encloses the compressible seal within said valve assembly, said rotatable locking nut being rotatable about said valve body to selectively exert and remove compressive force applied to said compressible seal so as to selectively close and open said passage thereof about the circumference of an intravascular instrument in response to the exertion and removal of said force respectively.

11. The hemostasis valve of claim 10, wherein said first connector is connected to said rotatable locking nut.

12. The hemostasis valve of claim 11, wherein said first connector is a luer lock female connector; and wherein said second connector is a rotatable luer lock male connector.

13. A hemostasis valve for preventing blood loss while permitting the introduction of intravascular instruments, the hemostasis valve comprising:
a valve body having a proximal end, a distal end, an intermediate barrel portion, and lumen extending through said valve body, said lumen capable of receiving for passage therethrough at least one intravascular instrument;
a first compressible seal having a normally open passage therethrough positioned within said valve body across said lumen, and which is selectively closeable when deformed by exerting a compressive force on the compressible seal;
a first rotatable locking nut joined to said valve body and forming a first valve assembly therewith which together encloses the first compressible seal within said first valve assembly, said first rotatable locking nut being rotatable about said valve body to selectively exert and remove compressive force applied to said compressible seal so as to selectively close and open said passage thereof about the circumference of an intravascular instrument in response to the exertion and removal of said force respectively;
a second compressible seal having a normally open passage therethrough positioned within said valve body across said lumen at a longitudinally spaced distance from said first compressible seal, and which is selectively closeable when deformed by exerting a compressive force on the compressible seal;
a second rotatable locking nut having a proximal end connected to said valve body, and a distal end connected to said intermediate barrel portion and forming a second valve assembly therewith which together encloses the second compressible seal within said second valve assembly;
said second rotatable locking nut being rotatable about said barrel portion to selectively exert and remove compressive force applied to said second compressible seal so as to selectively close and open said passage thereof about the circumference of an intravascular instrument in response to the exertion and removal of said force respectively;
said proximal end of said second rotatable locking nut being rotatably connected to said valve body for rotation about said valve body without effecting rotation of said second rotatable locking nut about said barrel portion;
a connector rotatably attached to said distal end of said valve body for connecting the hemostasis valve to another instrument; and
wherein each passage of said first and said second compressible seals are selectively and opened and closed to permit admittance of at least one intravascular instrument through at least one of said passage of said first and second compressible seals while maintaining hemostasis.
